(19) United States
(12) Patent Application Publication
(10) Pub. No.: US 2020/0241525 A1
(43) Pub. Date: Jul. 30, 2020

(54) Computer-based apparatus system for assessing, predicting, correcting, recovering, and reducing risk arising from an operator?s deficient situation awareness

(71) Applicant: Steven D. Harbour, Xenia, OH (US)

(72) Inventor: Steven D. Harbour, Xenia, OH (US)

(73) Assignee: Human Autonomous Solutions LLC, Dyaton, OH (US)

(21) Appl. No.: 16/396,648

(22) Filed: Apr. 27, 2019

Related U.S. Application Data
(60) Provisional application No. 62/797,335, filed on Jan. 27, 2019.

(51) Int. Cl. G05D 1/00; G06N 3/08; G06F 3/01; B60W 50/12; B60W 50/08; B60W 50/16; B60W 50/14; B60W 50/00; B60W 40/09; A61B 5/0478; A61B 5/00; A61B 5/18

(52) U.S. Cl. CPC G05D 1/0061; A61B 5/6823; G06F 3/015; G06F 3/014; G05D 1/0088; B60W 50/12; B60W 50/085; B60W 50/16; B60W 50/14; B60W 50/0097; B60W 40/09; A61B 5/0478; A61B 5/746; A61B 5/7264; A61B 5/18; A61B 5/681; A61B 5/6803; A61B 5/6893; A61B 5/7405; A61B 5/7455; A61B 5/0077; G05D 2201/0213; B60W 2050/143; B60W 2050/146; B60W 2540/22; B60W 2422/00; A61B 5/6816; A61B 5/6826; G06N 3/08

(57) ABSTRACT

A computer-based apparatus system for assessing, predicting, correcting, recovering, and reducing risk arising from an operator's deficient situation awareness (SA). This system identifies operator situation awareness by computer-apparatus and method that utilizes neurogenic-psychophysiological-neurocognitive-artificial intelligence processes. This system is configured to receive psychophysiological data from the operator via the neurogenic sensor(s) and configured to be loaded with data corresponding to the operator's baseline SA capacity and/or possesses AI algorithms to learn and calibrate the operator's baseline SA capacity and sound a warning in response if an SA deficiency threshold has been exceeded. Optionally, an autopilot/auto-driver/auto-operator/auto-worker/student alert/player & coach alert interface is configured to activate an autopilot/auto-driver/auto-operator/auto-worker command in response an SA deficiency threshold has been exceeded.

Pursuant to 37 C.F.R. § 1.78(a)(4), this Utility application claims the benefit of and priority to prior filed co-pending U.S. Provisional Patent: 62/734,258 filed 17 Sep. 2018, and U.S. Provisional Patent: 62/797,335 filed 27 Jan. 2019, which is expressly incorporated herein by reference.

### FIELD OF THE INVENTION

The present invention relates generally to the detection of an individual's situational awareness. More particularly, but not by way of limitation, the invention relates to situation awareness for determining risk in operating a vehicle, such as aviation (aircraft/ATC)/vehicle (auto/truck)/transportation (train/ship)/factory safety systems (machinery operation), one's ability to perform in an activity such as a students, players, and coaches and, more particularly, and to systems for monitoring and intervening during lapses in an operator's situation awareness (includes: pilot/driver/operator/worker/engineer/student, etc.).

### BACKGROUND OF THE INVENTION

Cognitive situation awareness (SA) and or Driver awareness (DA) and or Operator awareness (OA) has been a topic of research in the behavioral sciences for some time. SA can be DA, or OA, and may be defined as the human ability and capacity to perceive and comprehend the environment, and to use that information to accomplish a task. In more detail, SA in real-world terms for aviation/automobile/truck/train/ship/machinery, is the cognitive awareness level by the pilot/driver/operator as it relates to aircraft flight/vehicle-train-ship-machinery operation. Aircraft flight/vehicle-train-ship-machinery operation can be described by six degrees of freedom; therefore, an aspect of situation awareness is the cognitive awareness level by the individual of those six degrees. Those six degrees also move as a function of time; therefore, situation awareness also includes time and change awareness.

Situation awareness involves cognition and working memory, rather than action and response. It feeds on the perception of the elements in one's world within a volume of space and time. More specifically or to the point, SA is the psychological ability and capacity to perceive information and act on it acceptably. It has long been known that cognitive SA is critical to a broad range of human performance activities. Consequently, industry and academia are interested in improving SA of the user (e.g., by improving the man-machine interface via more efficient presentation of data to the user). In a complementary vein, improvements with respect to identifying lapses in SA, and presenting effective interventions thereto, are also subject of intense study.

Deficient Situation Awareness (SA) can easily lead to fatal accidents. To illustrate the severe consequences of such deficiencies of SA, it should be noted that the number-one killer in aviation is controlled flight into terrain (CFIT). Put simply, because of task saturation, inattentiveness, or sleepiness, failure to appropriately react to instrumentation and environmental cues, etc., the pilot/driver/operator/worker allows the aircraft to fly into the surface of the earth. Deficient Driver Awareness (DA) can easily lead to fatal accidents. To illustrate the severe consequences of such deficiencies of DA, it should be noted that “Driver Distraction”, “Task saturation”, “Inattentiveness”, “Lost in thought”, or “Driver Fatigue”, “Drowsiness”, “Falling Asleep” are leading causes of auto accidents. Put simply, because of task saturation, inattentiveness, drowsiness, falling asleep, failure to appropriately react to instrumentation and environmental cues, etc., the driver allows the car to collide into other cars, pedestrians, objects, and or veer off the prepared surface (losing control of the vehicle).

Obviously, the current loss to life and property affects society worldwide. This applies to all types of operators and factory workers as well.

It has been reported that if situation awareness could be improved, CFIT accidents could be reduced up to 73 percent, saving lives and an estimated $1 million per commercial and military aircraft over the next 10 years. Obviously, the current loss to life and property affects society worldwide. As a result, there exists an unmet need in the art for a system and method for Timely identifying deficient SA in a user, and thereafter triggering an appropriate intervention to mitigate the risk of harm that would result from continued operation of equipment under a deficient SA state. This applies to all types of operators and factory workers, as it does to students in a classroom who need to pay attention, and as it does to sports players and coaches as a team in a game.

Additionally, midair collisions are a continual threat, and can be caused by deficient SA. Of course, while the statistics and mode of harm are different, lapses of SA are frequently identified in after-action reports of bus and train collision catastrophes, distracted driving accidents, heavy equipment operation, and the like. Additional problems such as medications, e.g., medical marijuana add further risk. The number of drivers involved in fatal crashes in Colorado who tested positive for marijuana (after the fact—in hospital or morgue testing) has risen sharply (“federal and state data shows more than doubling each year since 2013 in that time”). Police in Colorado and California do not have an effective and definitive road side test to see if drivers are on marijuana. States need more exact testing as more States legalize marijuana's use.

The present invention provides a solution (such as a road side test kit) which is effective and definitive in testing a driver for the levels of influence due to marijuana. The National Highway Transportation Safety Association (NHTSA) estimates that drowsy and distracted driving were responsible for 72,000 crashes, 44,000 injuries, and 4,900 deaths in 2017 (NHTSA, 2018). However, these numbers are drastically underestimated and up to 6,000 fatal crashes, potential multiple deaths in each crash each year are caused by drowsy and distracted drivers according to the CDC. Deficient Situation Awareness (DSA) leads to fatal accidents for aircraft pilots/controllers/operators. The number one killer in commercial aviation is “CFIT”—Controlled Flight Into Terrain. It has been reported that if situation awareness is improved this accident potential would be reduced by 73 percent, saving lives and an estimated $150 Billion in aviation over the next 10 years. A CSX engineer who was operating a northbound train pulling 100 cars of phosphate fell asleep moments before it plowed into a southbound train pulling 110 cars of coal. In Train travel, operator fatigue accounts for the most recent lethal wrecks over the past decade.

Despite the importance of SA in human performance, in much of the study of the subject to date, there was no consensus on predictors or antecedent conditions (it was unpredictable). Rigorous study of SA was elusive and theoretically vague since it was unclear how to identify if a set of environmental conditions, cognitive processing load, and required user tasks would have a deleterious impact on one operator as compared to another operator under the same conditions.

As a result, there exists an unmet need in the art for a system and method for identifying deficient SA in an individual, and thereafter triggering an appropriate intervention to mitigate the risk of loss or harm that would result from the deficient SA state.

### SUMMARY OF THE INVENTION

It is an object to reduce the loss and risk associated with situational awareness.

A further object is to provide a computer based system to assist with detection of reduced situational awareness.

Accordingly, one aspect of the invention is directed to a computer-based apparatus system for assessing, predicting, correcting, recovering, and reducing risk arising from deficient situation awareness of an individual, said system including a processor having a memory, a neurogenic sensor operably disposed adjacent the individual to sense neurogenic and psychophysiological conditions of the individual and provide a neurogenic and psychophysiological data signal, a data acquisition module in electrical communication with the processor and configured to receive the neurogenic and psychophysiological data signal from the neurogenic sensor, and an individual variable module in electrical communication with said processor, wherein said individual variable module includes at least one of uploaded data corresponding to an individual's baseline situation awareness capacity and artificial intelligence and machine learning neural networks that learn and calibrate to an individual's baseline SA capacity and for determining if one or more predetermined situation awareness deficiency thresholds is exceeded, and a warning module in electrical communication with the processor configured to activate an alarm in response to the one or more predetermined situation awareness deficiency thresholds being exceeded.

The present invention overcomes the foregoing problems and other shortcomings, drawbacks, and challenges of preventing accidents resulting from an individual's decreased situation awareness (operator can also be pilot, driver, operator, worker, engineer, student, player, or coach). While the invention will be described in connection with certain embodiments, it will be understood that the invention is not limited to these embodiments. To the contrary, this invention includes all alternatives, modifications, and equivalents as may be included within the spirit and scope of the present invention. Vehicle as referred to herein can include, but not be limited to an automobile, bus, plane, truck, train, ship, machinery. Additionally, the invention has application to a classroom, a sport or a game activity.

The data acquisition module may include an electroencephalogram (EEG) apparatus onboard or coupled thereto, EEG processing of theta and alpha brainwaves in-operation as a neurogenic measure, which can additionally be used as augmentation to evaluate a pilot/driver/operator/student/team member/player/worker's cognitive state. Utilizing topographic EEG data indicates changes in the recorded patterns of alpha activity that are consistent with the mental demands of the various segments of the tasks being performed. Spontaneous EEG is conventionally classified into five clinical frequency bands, derived via Fourier transform of time-series EEG data: delta (0.5-4 Hz), theta (4-8 Hz), alpha (8-12 Hz), beta (12-31 Hz), and gamma (31-43 Hz). To determine a pilot/driver/operator/worker's cognitive state for use in this application of workload assessment and adaptive automation, the spontaneous EEG will be used, specifically theta and alpha for events from which event related activity is obtained and additionally this includes but not limited to, atrial or ventricular frequency and atrial or ventricular frequency variability and skin conductance) from the operator via the neurogenic sensor(s) ear worn, finger worn, in the seat, in control stick, in yoke, in steering wheel, in a watch, and or by camera; an operator variable module in electrical communication with the processor, wherein the operator variable module is loaded with data corresponding to the operator's baseline SA capacity and utilizes artificial intelligence and machine learning neural networks that can learn and calibrate to the operator's baseline SA capacity to ultimately determine which, if any, of the configured first or second thresholds should be deemed as exceeded during flight/drive/operation, the following: pilot/driver/operator/worker/driver/operator/student/player worker-specific relationships may be used during intermediate steps (AI Algorithms): Overall Predicted Z_SA=11.646−0.606Z_Vp−0.057Z_Va−0.334Z_Vswm, high task difficulty Predicted Z_SA=11.646−0.277Z_Vp−0.460Z_Va−0.055Z_Vswm, and Low task difficulty Predicted Z_SA=11.646+0.062Z_Vp−0.229Z_Va−0.530Z_Vswm. As discussed herein, Vp, Va, and Vswm scores, as well as and such as, but not limited to atrial periodic frequency and atrial periodic frequency variability and skin conductance (taken together, the pilot/driver/operator/worker-specific variable profile, or operator-specific variable profile) are obtained during the pilot/driver/operator/worker calibration steps and or learning steps. As a result, Values for Overall Predicted Z_SA, High Task Difficulty Predicted Z_SA, and Low Task Difficulty Predicted Z_SA are all available for use and comparison during operation conditions. An operator variable module is in electrical communication with the processor and configured to be loaded with data corresponding to the operator's baseline SA capacity. Wherein the operator variable module is also self-learned by way of artificial intelligence and machine learning neural networks with data corresponding to the operator's baseline SA capacity and normalized baseline and transient values may be captured prior to and during actual operations. A time versus voltage plot of cardio-electromotive forces may be obtained to ascertain the periodic atrial or ventricular frequency of the operator. For an individual not suffering from cardiac ailments, differences in the periodic ventricular or atrial frequencies will be inconsequential.

As a result, periodic atrial frequency and periodic ventricular frequency may be used interchangeably in the discussion that follows. Likewise, the periodic variability of either the atrial or ventricular frequencies can be calculated and stored. This includes but not limited to, atrial or ventricular frequency and atrial or ventricular frequency variability. A warning module in electrical communication with the processor and configured to activate a warning in response to the processor deciding that a first SA or DA deficiency threshold has been exceeded; and an auto-driver or autopilot/driver/operator/worker/interface in electrical communication with the processor and configured to activate an auto-driver or auto-driver command in response to the processor deciding that a second SA or DA deficiency threshold has been exceeded. An auto-driver interface is in electrical communication with the processor and is configured to activate an auto-driver command in response to the processor deciding that an SA deficiency threshold has been exceeded. A warning module, in electrical communication with the processor, is configured to activate a warning in response to the processor deciding that a first SA deficiency threshold has been exceeded. An autopilot/driver/operator/worker/interface is in electrical communication with the processor and is configured to activate an autopilot/driver/operator/worker/auto-driver command in response to the processor deciding that a second SA deficiency threshold has been exceeded. This invention employs three neurocognitive variables by the inventor that influence SA which also are correlated to changes in cognitive state which will lead to changes in sympathetic nervous system activity, with inversely proportional changes in parasympathetic nervous system activity which influence psychophysiological data when SA changes.

In accordance with another embodiment of the disclosed invention, a method for intervening during an operator's deficient situation awareness (SA) while operating a vehicle is provided. The method includes performing optional standardized cognitive tests to obtain variables correlating to the operator's baseline SA capacity; recording psychophysiological data from the operator while performing simulated tasks at a variety of different cognitive workloads. The method further includes generating an operator-specific variable profile corresponding to the operator's baseline SA capacity and psychophysiological data recorded while performing the tasks at the variety of different cognitive workloads. A first threshold corresponding to a first level of degraded SA is established, and a second threshold corresponds to a second level of degraded SA is established as well. Real-time psychophysiological data from the operator is monitored during the operation of the vehicle. The operator-specific variable profile and the real time psychophysiological data are used to decide when the operator has exceeded the first threshold. Likewise, the operator-specific variable profile and the real time psychophysiological data are used to decide when the operator has exceeded the second threshold. Or a first threshold corresponding to a first level of degraded SA is established, and a second threshold corresponds to a second level of degraded SA is established as well. Real-time psychophysiological data from the operator is monitored during the operation of the vehicle. The operator-specific variable profile and the real time psychophysiological data are used to decide when the operator has exceeded the first threshold. Likewise, the operator-specific variable profile and the real time psychophysiological data are used to decide when the operator has exceeded the second threshold.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a block diagram of an exemplary system, configured to interface with external peripherals, and to perform embodiments of the disclosed and claimed method.

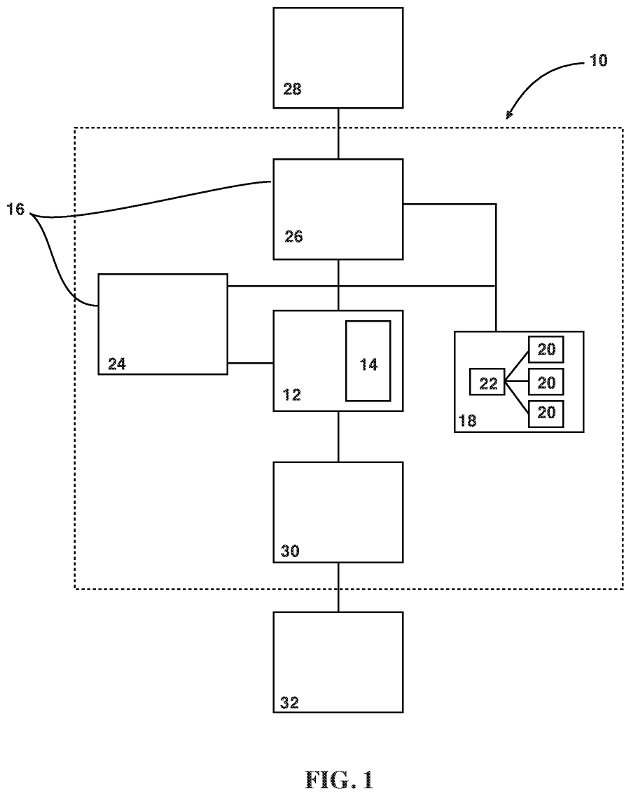

FIG. 2 depicts a flowchart representing steps of a method in accordance with embodiments of the disclosed invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

In the discussion that follows, specific reference will be made to an individual's particular activity, such as the interaction between a pilot/driver/operator/worker/engineer/and an aircraft/automobile/truck/ship/machinery. While the pilot/driver/operator/worker/-machine or driver-machine or operator-machine interface is one technology area that will greatly benefit from embodiments of the invention, such an operating environment is not intended to limit the scope of the disclosed and claimed invention. It will be recognized by one of ordinary skill in the art, that the disclosed invention may be adapted to operate in conjunction with automobiles, trains, personal watercraft, heavy-industrial equipment, and the like. Factors that will necessitate modifying the invention for use in non-aircraft environments will be discussed after the aircraft-centric method and apparatus, and the automobile-centric method and apparatus have been described. Additionally, it will be discussed that the invention covers commercial, government, and private operators in different ways and methods. Further, other activities include classroom and sport environment applications.

In an embodiment, operating an aircraft or automobile (car), a pilot/driver/operator/worker senses are assaulted with stimuli, and one must sense, acknowledge, process, and respond to a high percentage of those stimuli to ensure safe operation of the aircraft or vehicle. As one can imagine, the harm that may result from a failure to appropriately respond to a given stimulus will vary greatly. By way of example, some cockpit indicators appraise the pilot/driver/operator/worker of operating conditions that are innocuous, while others indicate a condition that requires immediate correction to maintain controlled flight. Additionally, oftentimes a plurality of stimuli must be processed to gain context to the severity of the issue at hand. By way of example, a thud perceived by the pilot/driver/operator/worker/driver/operator/worker, while flying in a non-austere environment, could be the result of any number of things.

A thud, in conjunction with an engine trouble indicator, might be the result of a bird strike, foreign object damage, or the like. As a result, since the pilot/driver/operator/worker/does not have the luxury of hindsight to inform him of the criticality of a given stimulus, he must do everything in his power to main SA with respect to the greatest number of stimuli as possible. By way of example, some car instrument panel indicators appraise the driver of operating conditions that are innocuous, while others indicate a condition that requires immediate correction to maintain control of the car. Additionally, oftentimes a plurality of stimuli must be processed to gain context to the severity of the issue at hand. By way of example, texting or talking on the cell phone even if it is handsfree and driving, can be taxing to the driver causing SA or awareness to greatly degrade. Couple that while motoring in a heavy traffic environment, or it could be the result of any number of things, could and often times lead to disaster. Or if the driver is bored and or daydreaming or complacent, or fatigue or drowsy or worse falling asleep the results can be the same and that is a crash result with fatalities. As a result, since the driver does not have the luxury of hindsight to inform him of the criticality of a given stimulus, he must do everything in his power to maintain SA with respect to the greatest number of stimuli as possible or stay alert and awake and not fall asleep either.

A number of circumstances may lead to degraded SA. Receiving too many simultaneous stimuli, performing complex hand eye manipulations in parallel with decision-making tasks, high cognitive workload alone, boredom, fatigue, and combinations or permutations thereof can all contribute to loss of SA. While degraded SA actually occurs in a quasi-continuously variable manner, systems using discrete SA degradation values are useful (e.g. China Lake Situation Awareness (CLSA) Score, Bedford Workload Scale, and other assessment scales).

As will be explained herein, an embodiment of the disclosed invention uses a processor having memory configured with data corresponding to calibrated attributes of a particular pilot/driver/operator/worker/driver/operator/student/player and or through artificial intelligence methods utilizing a neural network to learn and fine and improve upon the stored attributes of a particular pilot/driver/operator/worker/player/student. The processor receives data from psychophysiological sensors coupled to the pilot/driver/operator/worker/student/player. In addition to the/operator/worker/driver/operator calibration and psychophysiological data, the processor includes one or more threshold levels, wherein each threshold level corresponds to a given SA level. The processor is configured to interface with other systems, and in response to a detected threshold being exceeded, provide an appropriate output and cooperating corrective action. The threshold may also be referred to as a SA deficiency threshold. Cognition is the human process that is mental activity, which utilizes thoughts for acquiring, processing, storing, transforming, and using knowledge. Perception involves unaware inference utilizing both biological and psychological processes. Items and or events in the environment give off clues to their existence, and the sensory organs detect these clues. Vision is one of the five senses and the most involved when interfacing with an operation of a vehicle/car/truck/plane/train/ship/classroom learning/playing sports game. The raw beginnings of perception begin with visual sensation, first a stimulus occurs. The human typically first mentally performs visual bottom-up processing then a fraction of a second later visual top-down processing. Memory is the storage of information, either sensory memory, short-term (working memory) or long-term.

Mental workload is related to both demand and cognitive capacity. The demand is imposed by tasks on the human's limited mental resources, whether considered as single or multiple. However, more specifically mental workload for this application is an assessment of what proportion of mental capacity is demanded by a task. Mental workload can be measured as an implicit measurement of situation awareness (SA). Speed-working memory (SWM) is the reaction time-frame that includes the processes of memory, visual scanning, and perception. The speed of responses is measured in milliseconds to stimuli for cognitive decisions to occur. Psychophysiological measurements are listing as the following but are not limited to the following:

Electroencephalography (EEG), as a psychophysiological measure, has been successfully used to estimate an operator's cognitive state. First studied in animals by Richard Catton in 1875, electroencephalography is the study of the electrical activity of the brain. It has been determined that superposition of post-synaptic potentials due to volume conduction causes the observable electric activity at or within 0.5 inches from the scalp or other potential on body locations. Topographic EEG data will show changes in the recorded patterns of alpha activity that are consistent with the mental demands of the various segments of the tasks being performed. There are two ways to study EEG: time-locked to the stimulus (event-related or evoked potential) or spectral (windowed/averaged in time). Without an external stimulus, spontaneous EEG occurs, such as alpha and beta rhythms, whereas ERPs occur in response to a specific stimulus. Spontaneous EEG is classified into five clinical frequency bands, derived via Fourier transform of time-series EEG data: delta (0.5-4 Hz), theta (4-8 Hz), alpha (8-12 Hz), beta (12-31 Hz), and gamma (31-43 Hz). To determine a person's cognitive state for use in applications such as workload assessment and adaptive automation, the spontaneous EEG should be used, even without specific events from which ERPs are obtained. During mentally demanding portions of tasks frontal theta-band EEG has shown increased activity, therefore, theta-band EEG should increase as mental workload increases. Topographic EEG data from channels illustrates decreases in parietal alpha-band activity, which correlated to higher mental demands therefore, parietal alpha-band EEG should decrease as mental workload increases. Multiple EEG recording sites are useful to detect significant changes in regional brain activity that are related to different tasks. It has been found that increases in cognitive workload were associated with decreased EEG alpha-band power over parietal sites, with corresponding increases in theta power. Therefore, theta and alpha diverge, when mental workload significantly increases, consequently indicated of decreasing SA.

Psychophysiology measurements including the combination of ECG and EEG have shown a high level of accuracy and validity in explicitly measuring workload which in turn is implicitly measuring situation awareness. As mental workload increases up to a certain point, then cognitive situation awareness decreases, as workload continues to increase, consequently, as one measures the current psychological and physiological state of an individual, one is measuring mental workload and in turn, cognitive situation awareness. Electrocardiography is a method used to analyze the myocardium's conduction system, which provides us with information about the myocardium's electrical activity (the myocardium rhythm and activity). The recording of the conduction system is physically represented as an ECG. The P wave represents the depolarization (contraction) of the atria, the PR segment the transmission of the electrical impulse to the ventricles, the QRS complex represents depolarization (contraction) of the ventricles and the T wave shows the repolarization (relaxation) of the ventricles. Detecting the small electrical changes that are a consequence of cardiac muscle depolarization followed by repolarization during each cardiac cycle give us a measure of atrial or ventricular frequency. Segments during this can be analyzed by extracting the average measure atrial or ventricular frequency and standard deviation of the ventricular or atrial frequency interval yields ventricular or arterial frequency variability as a measure. It is noted that this is a psychophysiological measurement as well, which is objective, provides more granularity and significant differences in workload as a function of task.

These objective measures from EEG and ECG are part of psychophysiological measurements. Impedances should be below 40 kilo-ohms for accuracy from sensor. Extracting data segments cardio data is band-pass filtered from 0.4 Hz to 30 Hz. Cardio wave peaks are marked using QRS tool based on threshold detection followed by inter-frequency intervals during processing by way of Fourier Transform analysis. The cardio rhythmic activity is influenced by higher-order brain centers. Cognitive activity will change the rhythmic patterns; therefore, as cognitive activity occurs, such as mental workload, it causes a change in atrial ventricular frequency and variability. Increased mental workload will lead to an increase in sympathetic nervous system activity, and a decrease in parasympathetic nervous system activity for cardio regulation. Consequently, atrial ventricular frequency increases as mental WL increases and cardio atrial ventricular frequency variability decreases as WL increases—with an indirect influence on SA. This changes also occur for “sleepiness”, “inattention”, “distraction”, “task saturation”, and “marijuana use” and other “medications such as opioids”. Sleep is another psychophysiological measure; sleep deprivation is indicative of and individual who will be more prone to deficient SA and Blood Pressure will lower during sleepiness and rise during stress. Body Temperature will also lower during sleepiness and rise during stress making it very useful as a psychophysiological measure for SA. Skin conductance and Blood Oxygen levels change with alertness and affect SA.

The invention provides for use of three neurocognitive factors; visual attentiveness (Va), visual perceptiveness (Vp), and visuospatial working memory (Vswm) as part of this inventions process for obtaining individual SA. These neurocognitive factors integrated with the psychophysiological data yield the individual composite SA assessment and predictive levels. Based on a model that conceptualized attention as a multidimensional capacity that includes five primary factors including focused, sustained, selective, alternating and divided attention. Visual Attentiveness is derived from visual Vigilance, Focus, and Speed, involving both static and dynamic visual processing. Visual vigilance is the maintenance of attention required to respond to a change in the environment (a state of inhibition) such as responding to a target. Intuitively, the mind can wander; the subject must maintain his or her attention in order not to miss a target. Visual focus is the consistency (lack of variance) in reaction time to a change in the environment and reflects the sustaining and maintaining of attention. Visual speed is defined as the average reaction time to changes in the environment. This measures discriminatory mental processing speed. Both are measured in milliseconds. To elaborate, Visual Attentiveness (Va) is the ability to concentrate and be devoted, to be diligent and detailed, alert, watchful, and responsive. Visual perceptiveness is similarly a composite score based on three sub-scores. It consists of visual Prudence, Consistency, and Stamina and involves both static and dynamic visual processing. Visual prudence is the selection or choice of a correct response or responses in the environment (non-inhibited). That is being non-impulsive, the ability to not automatically react yielding an incorrect response in the changing environment. Visual consistency is the ability to stay on task, respond reliably, making dependable responses in a dynamic environment. Visual stamina is the lack of variability in a subject's response times in the environment. This is the ability to maintaining a sustained effort over time. These are measured in milliseconds. To elaborate, Visual Perceptiveness (Vp) is the ability to have a perceptivity, to be insightful, and to have discernment—the ability to perceive that which is obscured. For salience, Visual Perceptiveness is visual insightfulness or perceptivity and it is equivalent to: Prudence as a measure of Carefulness (antiimpulsivity) and response inhibition as evidenced by three different types of errors of commission (seeks traits of consisting of being circumspective and mindful). Consistency, which measures the general reliability and variability of response times and is used to help measure the ability to stay on task, and Stamina, which compares the mean reaction times of correct responses during the first 200 trials to the last 200 trials. This score is used to identify levels of sustaining attention and effort over time.

Visuospatial Working Memory (IV) is working memory that contains visual and spatial information that is stored in the visuospatial sketchpad in the mind, this immediate memory can be thought of as a workbench where material is continuously being combined and transformed. Visuospatial working memory was assessed via the spatial n-back task, already explained in detail in this section along with its validity. This variable's level of measurement is ratio and is a percent of selections that are correct out of the total number of trials. Scores can range from 0% correct to 100% correct. These are inherent neurocognitive factors. Neuroergonomics is a rapidly expanding, interdisciplinary area of research whose purpose is to enhance knowledge of brain activity, function, and human behavior as encountered at work and in natural settings. It is integrated research between psychology, cognitive neuroscience, human factors, engineering, computer science, ergonomics, and medicine. For example, neuroergonomics can focus on the psychology of perceptual and cognitive functions and actions in relation to actual technologies. A premise of this multidisciplinary approach is that knowledge in the psychological sciences will be enhanced. Neuroergonomics is the brain at work indicated which is a composite exploration and is growing and is being driven by the emergence of information-saturated information display technology that is now being utilized by humans for activities requiring divided attention and multitasking. Neuroergonomics provides a novel approach as a contemporary perspective in science, and field research of human situation awareness is an integral part. Neurogenic: is arising from the nerves or the nervous system. Neurocognitive functions are cognitive functions closely linked to the function of particular areas, neural pathways, or cortical networks in the brain substrate layers of neurological matrix at the cellular molecular level.

An embodiment of the invention will first be described as a method. Thereafter, a system suitable for performance of the method will be disclosed. It will be recognized by one of ordinary skill in the art, that while the method is described in a particular order, embodiments of the method may omit some steps during certain iterations (by way of example and not limitation, omitting a calibration step), or certain portions of the method may be looped until a given result is achieved (perhaps for a given flight or drive or operation, an intervention by the system will not be required at all).

In accordance with an embodiment of the disclosed invention, a method of detecting decreased pilot/driver/operator/worker/driver/operator/worker Situation Awareness (SA) begins by exploring the baseline cognitive, visual, auditory, and/or neuromuscular skills of a particular pilot/driver/operator/worker. For example, standardized computer-based and, or written instruments may be used to assess the relative ability of a pilot/driver/operator/worker/driver/operator/worker to receive, process, and appropriately respond to given stimuli. The IVA+TM, N-back, and other instruments may be used to quantitatively assess a pilot/driver/operator/worker/driver/operator/worker's baseline SA ability. By way of example, one pilot/driver/operator/worker/driver/operator/worker may be capable of receiving stimulus inputs, while being able to recall data presented to him four cycles in the past. Conversely, another pilot/driver/operator/worker/may only be able to consistently recall data from two cycles in the past. The invention employs Variables Vp, Va, and Vswm, corresponding to perceptiveness visual ability, attentiveness visual ability, and spatial working memory visual ability, respectively, may be obtained by use of the previously discussed baseline tests. These variables or coefficients will be used in the steps of the computer based system and method that follow.

Other pilot/driver/operator/worker baseline values may be captured prior to actual flight or operation. A time versus voltage plot of cardio-electromotive forces may be obtained to ascertain the periodic atrial or ventricular frequency of the pilot/driver/operator/worker. For an individual not suffering from cardiac ailments, differences in the periodic ventricular or atrial frequencies will be inconsequential. As a result, periodic atrial frequency and periodic ventricular frequency may be used interchangeably in the discussion that follows. Likewise, the periodic variability of either the atrial or ventricular frequencies can be calculated and stored. This includes but not limited to, atrial or ventricular frequency and atrial or ventricular frequency variability.

Each of the periodic atrial or periodic ventricular frequencies, as well as the periodic variability thereof, should be captured under simulator conditions that artificially apply stimuli ranging from trivial task saturation (no loss of SA), moderate task saturation (moderate loss of SA), and severe task saturation (high loss of SA). The collection of Vp, Va, and Vswm, atrial periodic frequency (or ventricular periodic frequency), and atrial periodic frequency variability (or ventricular periodic frequency variability) may be referred to as a pilot/driver/operator/worker/-specific variable profile.

Additionally, the pilot/driver/operator/worker/driver/operator/worker's cranial alpha and theta wave activity may be evaluated. During mentally demanding tasks, front theta wave activity increases. Conversely, parietal alpha activity has been demonstrated to decrease during the processing of demanding cognitive tasks. As a result, divergence in Alpha and Theta activity is indicative of increasing mental workload.

This pilot/driver/operator/worker calibration steps are important because one pilot/driver/operator/worker may become dangerously task-saturated under a given workload, and exhibit differing psychophysiological changes, while another may tolerate an analogous workload and remain in safe control of the aircraft. It should be noted that this baseline SA ability may be improved through cognitive exercises (or perhaps degraded by physical injury, skill-atrophy, or other circumstances) and the baseline should be re-evaluated if there is reason to believe that the baseline may have changed for a given pilot/driver/operator/worker. While the IVA+TM, N-back test are useful to describe the subject invention, other tests having acceptable reliability and sensitivity can be employed.

The next step is to determine, for a given role or operating environment, which levels of degraded SA require identification or intervention. In the aforementioned Bedford Workload Scale, mental effort required to satisfy a given workload is evaluated on a scale of 1-10. Pilot/driver/operator/worker/driver/operator/workers are asked to self-assess (after completion of the flight) whether 1) it was possible to complete a task, 2) whether the workload was tolerable, and 3) if the workload was satisfactory without reduction. The ultimate rating of 1 corresponds to an appraised insignificant workload, and 10 corresponds to task abandonment. Likewise, the CLSA Scale uses 5 increments to connote very good SA at 1 and very poor SA at 5. While both assessments are known to have good sensitivity and reliability when applied to AF pilot/driver/operator/worker/driver/operator/workers in customary operating environments, the 5-point China Lake Score will be used in the following discussion. It is generally accepted that a CLSA Score of 3 is within the bounds of safe SA, and a CLSA score of 5 corresponds to nearly complete loss of SA. As a result, exemplary embodiments of the disclosed invention contemplate a first threshold corresponding to CLSA Score of 4, a second threshold corresponding to a CLSA Score of 5. Later, the discussion will explore how exceeded thresholds of degraded SA may be used to implement corrective actions.

To ultimately determine which, if any, of the configured first or second thresholds should be deemed as exceeded during flight, the following pilot/driver/operator/worker/-specific relationships may be used during intermediate steps (AI Algorithms): Overall Predicted Z_SA=11.646−0.606Z_Vp−0.057Z_Va−0.334Z_Vswm, high task difficulty Predicted Z_SA=11.646−0.277Z_Vp−0.460Z_Va−0.055Z_Vswm, and Low task difficulty Predicted Z_SA=11.646+0.062Z_Vp−0.229Z_Va−0.530Z_Vswm. As discussed above, Vp, Va, and Vswm scores, as well (but not limited to) atrial periodic frequency and atrial periodic frequency variability (taken together, the pilot/driver/operator/worker/-specific variable profile, or operator-specific variable profile) were all obtained during the pilot/driver/operator/worker/ calibration steps. As a result, Values for Overall Predicted Z_SA, High Task Difficulty Predicted Z_SA, and Low Task Difficulty Predicted Z_SA are all available for use and comparison and includes but not limited to, atrial or ventricular frequency and atrial or ventricular frequency variability.

During flight/Drive/Operation, the method makes a current SA state calculation that is updated at a pre-determined time interval (values of 0.5 to 6 seconds have been shown to produce acceptable results). The Current SA state calculation uses the individual pilot/driver/operator/worker/driver/operator/worker's “Overall Predicted SA score (Predicted Z_SA)” that is then cross compared against their “High task difficulty or Low task difficulty Predicted SA score (Predicted Z_SA)” as appropriate and the greater of the three values will be the initial point SA score under nominal conditions.

To that initial point SA score, the absolute value of the real time normalized change in atrial periodic frequency variability (This includes but not limited to, atrial or ventricular frequency and atrial or ventricular frequency variability) for that pilot/driver/operator/worker/driver/operator/worker, scaled as a SA score, is then added. When that “Total Resultant SA Score” exceeds the equivalent of a “4” on the combined CLSA scale, the first threshold is deemed to be exceeded. When this first threshold is exceeded, the pilot/driver/operator/worker/driver/operator/worker may be alerted by a visible WARNING message, audible tone, audible spoken message, electrostatic shock, or other indicator known to one of ordinary skill in the art. Furthermore, when the “Total Resultant SA score” is a “5,” the second threshold is deemed to be exceeded. When the second threshold is exceeded, an autopilot/driver/operator/worker/ command is invoked, thus taking control of the aircraft to auto-roll the aircraft to maintain level flight on a predetermined heading and altitude until the pilot/driver/operator/worker/driver/operator/worker's SA score returns to a “3” or lower. In certain embodiments, the normalized absolute value of the operator's atrial or ventricular frequency variability may compared with about e.g. 71.63 milliseconds to about e.g. 45.17 milliseconds yielding about e.g. 10.92 milliseconds per SA (Composite Score) Interval Level normalized. In other embodiments, wherein the first threshold normalized absolute value of the operator's atrial or ventricular frequency variability is about e.g. 34.25 milliseconds or 130.5 milliseconds respectively (e.g. a 40-45% reduction—due to sleepiness or distraction, or increase—due to marijuana use for roadside kit respectively), and the second threshold normalized absolute value of the operator's atrial or ventricular frequency variability is about e.g. 23.33 milliseconds or 140.0 milliseconds respectively (e.g. a 55-60% reduction—due to sleepiness or distraction, or increase—due to marijuana use for roadside kit respectively). The threshold normalized absolute value of the operator's atrial or ventricular frequency variability is about e.g. 34.25 milliseconds, and the second threshold normalized absolute value of the operator's atrial or ventricular frequency variability is about e.g. 23.33 milliseconds.

It will be recognized by one of ordinary skill in the art that the number of thresholds employed, as well as the points of demarcation thereof, may be varied to meet design objectives. In another embodiment, the pilot/driver/operator/worker/ may be required to take a quick calibration test prior to flight/drive/operations on a given day. A temporary SA correction factor may then be applied to his pilot/driver/operator/worker/-specific variable profile to temporarily modify the threshold points for a CLSA score of 4 or 5 if the pilot/driver/operator/worker/ is fatigued or otherwise impaired on a particular flying/driving/operation day.

In yet another embodiment, the action taken in response to exceeding a given threshold may be varied to a different pre-configured action, a user-selectable action, or a different selected action that is automatically manipulated by an external triggering event or signal. By way of example and not limitation, the action invoked upon exceeding the second threshold, in the example above, may be user-selected to be different under aerial combat conditions. If the pilot/driver/operator/worker elects to disable the autopilot/driver/operator/worker activation function, crossing the second threshold my result in a louder tone, haptic feedback, strobing lights, or the like, in lieu of invoking an autopilot/driver/operator/worker command. Likewise, invoking an “armed” status on a weapon control system may modify the action in an automated manner (inferring aerial combat in the absence of a distinct user-selection). Under such aerial combat conditions, it may be determined that the risk of harm from an erroneous invocation of auto-pilot/driver/operator/worker/ is unacceptably high. The different pre-configured action may lastly vary as a function of the operator's role (co-pilot/driver/operator/worker/, Weapon System Officer, Electronic Weapon system Officer, etc.).

The following example illustrates a particular system configured to exploit advantages of some of the embodiments of the present invention. Furthermore, the disclosed system is but one example of reduction to practice of the present invention. It serves as confirmation that the principles described in the present invention are therefore valid but should not be construed as in any way limiting the scope of the invention. A method for intervening during an operator's deficient situation awareness (SA) while operating a vehicle/aircraft/ship/machinery, the method comprising: performing standardized cognitive tests to obtain values of inventor discovered neurocognitive variables correlating to the operator's baseline SA capacity and or through artificial intelligence neural networks learning and calibrating to the operator's baseline SA capacity;

recording psychophysiological data from the operator while performing simulated tasks and or calibration at a variety of different cognitive workloads;
generating an operator-specific variable profile corresponding to the operator's baseline SA capacity and psychophysiological data recorded while performing the tasks at the variety of different cognitive workloads;
establishing a first threshold corresponding to a first level of degraded SA, and establishing a second threshold corresponds to a second level of degraded SA;
monitoring real-time psychophysiological data from the operator during the operation of the vehicle/aircraft/machinery;
using the operator-specific variable profile and or through artificial intelligence neural networks learning and calibrating to the operator-specific variable profile and the real time psychophysiological data to decide when the operator has exceeded the first threshold; and using the operator-specific variable profile and or through artificial intelligence neural networks learning and calibrating to the operator-specific variable profile and the real time psychophysiological data to decide when the operator has exceeded the second threshold. The operator-specific variable profile includes an overall predicted SA variable, a high task difficulty predicted SA variable, and a low task difficulty predicted SA variable.

If the operator has exceeded a predetermined threshold which includes comparing the normalized absolute value of the operator's atrial or ventricular frequency variability with a SA composite score interval level normalized, a warning signal or intervening signal can be generated to perform a desired function in the system.

The method of wherein the normalized absolute value of the operator's atrial or ventricular frequency variability is compared with about e.g. 71.63 milliseconds to about 45.17 milliseconds yielding about e.g. 10.92 milliseconds per SA (Composite Score) Interval Level normalized. The method, wherein the first threshold normalized absolute value of the operator's atrial or ventricular frequency variability is about e.g. 34.25 milliseconds or 130.5 milliseconds respectively (e.g. a 40-45% reduction—due to sleepiness or distraction or task saturation, or increase (e.g., due to marijuana use and other medications for roadside kit, respectively), and the second threshold normalized absolute value of the operator's atrial or ventricular frequency variability is about e.g. 23.33 milliseconds or 140.0 milliseconds respectively (e.g. a 55-60% reduction—due to sleepiness or distraction, or task saturation, or increase—due to marijuana or medicine use for roadside kit, respectively). The method, can also be contained in a road side kit for Law Enforcement and or integrated with Smart Phone and wireless technology via Blue Tooth technology and Smart Watch and or Satellite via communication link technology to receive and transmit that information (i.e. warning activation and or autopilot/auto-driver/auto-operator/auto-worker command activation).

An example of the invention is provided in the following embodiment. A system 10, can be for use in an aircraft/car/truck/ship and in conjunction with a pilot/driver/operator/worker/driver/operator/worker's operation thereof, is provided to implement the above disclosed method, and variants thereof. The system 10 includes a processor 12 having a memory 14. A plurality of modules 16 may be coupled to the processor 12, or the modules 16 may be emulated within the processor 12 in a virtual state.

A data acquisition module 18 includes a plurality of galvanometers 20 configured to be electrically coupled to the pilot/driver/operator/worker/ via leads and conductive pads known to those of ordinary skill in the art (not shown). Some embodiments of the disclosed invention may use a break-away connector between the pilot/driver/operator/worker/ and the system 10 to facilitate unencumbered egress from the aircraft in the event of ejection seat activation. Other embodiments envision use of wireless telemetry to couple the leads and conductive pads of the pilot/driver/operator/worker/ to the system 10. In yet another embodiment, galvanometers may be replaced with a reflective or transmissive photoplethysmography apparatus, and thereafter coupled to a single location on the pilot/driver/operator/worker's body. Again, the aforementioned transmissive or reflective photoplethysmography apparatus can be particularly suited for collecting psychophysiological data.

Some but not all, of the fitness data, that are frequently collected and incorporated into popular fitness tracking wristbands, can also be employed as psychophysiological data and those fitness tracking bands may share data with the system 10 for use in assessing SA. That data can include, but not limited to: skin conductance, plurality of galvanometers capturing a time versus voltage plot of the cutaneous-detected cardio-electromotive forces, ascertaining of the periodic atrial or ventricular frequency and or atrial or ventricular frequency variability, blood oxygenation, body temperature, blood sugar, blood pressure, and sleep. Consequently, such may be collected by fitness wristbands and incorporated and transmitted wireless to a multipurpose smart phone. Wherein, a smart phone application derived and made by the inventor contains aforementioned algorithms, methods, and processes, utilizes a portion or portions of that fitness wristband collected data, produces in a proprietary fashion an audible alert, alarm, warning when the individual's SA has become deficient. This smart phone application for use while operating a vehicle, a student in an educational setting such as a classroom, a participant in a sport or game player in a game. Allowing the public with an older technology vehicle/plane/train/ship/machinery who also owns a fitness wristband, and a smart phone, some protection against deficient SA, e.g. while driving, if they possess this technology contained herein in a smart phone application.

The plurality of galvanometers 20 are coupled to the processor 12 by way of an analog to digital converter (ADC) 22. The ADC 22 is configured to sample the analog values of the galvanometers 20 at a rate sufficient to capture a time versus voltage plot of the cutaneous-detected cardio-electromotive forces of the pilot/driver/operator/worker/. Under most operating conditions, an ADC 22 sampling rate from about 250 Hz to about 500 Hz has been shown to produce acceptable results. The time versus voltage plot may be used to ascertain the periodic atrial or ventricular frequency of the pilot/driver/operator/worker/driver/operator/worker.

The data acquisition module may also include an electroencephalogram (EEG) Apparatus onboard or coupled thereto. EEG processing of theta and alpha brainwaves in-flight as a neurogenic measure, can additionally be used as augmentation to evaluate a pilot/driver/operator/worker/driver/operator/worker's cognitive state. Utilizing topographic EEG data indicates changes in the scalp- recorded patterns of alpha activity that are consistent with the mental demands of the various segments of the tasks being performed. Spontaneous EEG is classified into five clinical frequency bands, derived via Fourier transform of time-series EEG data: delta (0.5-4 Hz), theta (4-8 Hz), alpha (8-12 Hz), beta (12-31 Hz), and gamma (31-43 Hz). To determine a pilot/driver/operator/worker/cognitive state for use in this application of workload assessment and adaptive automation, the spontaneous EEG will be used, specifically theta and alpha for events from which event related activity is obtained.

A pilot/driver/operator/worker/ variable module 24 is in electrical communication with the processor 12. In some embodiments, the module may be referred to as an operator variable monitor. It may include volatile or non-volatile storage and is configured to hold the pilot/driver/operator/worker/-specific variable profile. The input module 24 may remain in communication with the processor 12 for the duration of the flight/drive/ operation, or it may serve as a pre-flight/pre-drive/pre-operation interface to transfer the pilot/driver/operator/worker/driver/operator/worker-specific variable profile to the memory 14. It may be selectively coupled and de-coupled using multi-pin coaxial, universal serial bus, or other connection known to one of ordinary skill in the art.

The system 10 further includes a warning module 26 coupled to the processor 12 and configured to activate indicators in response to receiving a “first threshold exceeded” signal from the processor 12. In some embodiments, the warning module 26 is a standalone unit including audible, visual, or haptic indicators (that serve as warnings) coupled thereto. In other embodiments, the warning module 26 is an interface coupled to existing vehicle, such as an aircraft warning systems 28, that is configured provide compatible signals to activate indicators of the existing aircraft warning system 28. The warning module 26 can be contained in road side kit for law enforcement with the warning module operably coupled to the law enforcement warning system, and configured to activate an audible indicator, a visual indicator, and or a haptic indicator warning for law enforcement use.

An autopilot/driver/operator/worker/ interface 30 is coupled to the processor 12 and the aircraft flight control 32. In some embodiments, the aircraft flight control 32 is a Ground Proximity Warning System (GPWS), and in other embodiments the flight control 32 is the aircraft's autopilot/driver/operator/worker/ system. In either configuration, the autopilot/driver/operator/worker/ interface 30 responds to a “second threshold exceeded” signal from the processor 12, and thereafter provides compatible signals to the aircraft's autopilot/driver/operator/worker/ system or to the GPWS sufficient to reacquire controlled level flight/drive/boating/operation of the plane or car or truck or ship or machinery. The processor 12 is configured to use the pilot/driver/operator/worker/-specific variable profile stored in the pilot/driver/operator/worker/ variable module 24 or the memory 14, and calculate a corresponding Overall Predicted Z_SA, High Task Difficulty Predicted Z_SA, and Low Task Difficulty Predicted Z_SA.

During flight/drive/operation, the processor 12 makes a current SA state calculation that is updated at a pre-determined time interval (values of 1 to 5 seconds have been shown to produce acceptable results). The Current SA state calculation uses the individual pilot/driver/operator/worker/“Overall Predicted SA score (Predicted Z_SA)” that is then cross compared against their “High task difficulty or Low task difficulty Predicted SA score (Predicted Z_SA)” as appropriate and the greater of the three values will be the initial point SA score under nominal conditions.

To that initial point SA score, the absolute value of the real time normalized change in atrial periodic frequency variability profile for that pilot/driver/operator/worker/, scaled as an SA score, is then added. When that “Total Resultant SA Score” exceeds the equivalent of a “4” (predetermined threshhold) on the combined CLSA scale, the first threshold is deemed to be exceeded. When this first threshold is exceeded, the processor 12 generates a first threshold exceeded signal to the warning module 26. The warning module 26 can be by way of this example configured to generate an indicator or configured to active an indicator of the existing aircraft warning system 28.

Furthermore, when the “Total Resultant SA score” is a “5” the second threshold is deemed to be exceeded. When the second threshold is exceeded, the processor 12 generates a second threshold exceeded signal to the autopilot/driver/operator/worker/interface 30. The autopilot/driver/operator/worker/interface 30 is configured to communicate with the aircraft flight controls 32 and issue an autopilot/driver/operator/worker/command to auto-roll the aircraft to maintain level flight on a predetermined heading and altitude until the pilot/driver/operator/worker/SA score returns to a “3” or lower.

Embodiments of the disclosed invention may be adapted for use with operators of other types of equipment. For example, when used with an automobile, the system 10 may interface with the CAN bus or other serial interface of the vehicle to activate the vehicle's hazard signals, horn, instrument cluster notification, audio system, pulse the brakes, reduce throttle, or activate other systems in response to exceeding a threshold value of SA degradation.

In some embodiments of the disclosed invention, data relating to a particular driver's baseline SA variables may be encoded in the vehicle's key and transmitted to the vehicle during a request for ignition. Similarly, some embodiments may use a vehicle's existing security key transponder serial number to indicate a driver's identity, and thereafter recall that driver's variables for use during operation.

The aforementioned transmissive or reflective photoplethysmography apparatus is particularly suited for use in vehicular applications. Such apparatus may be placed into a dedicated wristband for use while driving or may be incorporated into a wireless multipurpose audio earbud. Further, reflective photoplethysmography apparatus are frequently incorporated into popular fitness tracking wristbands, and those fitness tracking bands may share data with the system 10 for use in assessing a driver's SA.

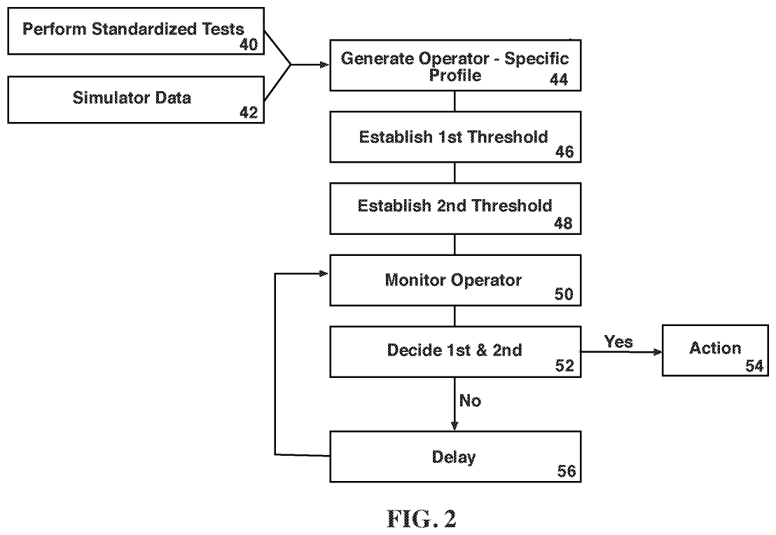

Turning attention to FIG. 2, the combined system and method may be represented by the following provided flowchart. At block 40 (Perform Standardized Tests), personnel administer standardized cognitive tests to the operator to obtain variables correlating to the operator's baseline SA capacity. At block 42 (simulator data), personnel record psychophysiological data from the operator while the operator performs a variety of cognitive workloads in an aircraft or vehicle simulator. Block 40 and 42 are used to generate an operator-specific variable profile corresponding to the operator's baseline SA capacity and psychophysiological data recorded while performing the tasks at the variety of different cognitive workloads at block 44 (generate operator-specific variable profile) and or AI machine learning is achieved. A first threshold corresponding to a first level of degraded SA is established at block 46, and a second threshold corresponds to a second level of degraded SA is established at block 48. The real time psychophysiological data of the operator is monitored during the operation of the vehicle at block 50 (monitor pilot/driver/operator/worker/). Using the operator-specific variable profile and the real time psychophysiological data to decide when the operator has exceeded the first threshold and the second threshold at block 52 (Decide 1st and 2nd). If the first threshold or the first and the second threshold have been exceeded, an action is performed at block 54.

That action may be a warning, an autopilot, driver, operator, worker, command, or other appropriate action given the type of equipment being operated. If neither threshold is exceeded, a delay at block 56 is optionally used before iteratively monitoring the pilot, driver, operator, worker, again. In the absence of the delay block 56, monitoring of the pilot, driver, operator, worker would occur immediately.

Another aspect of the invention contemplates the computer-based apparatus system for assessing, predicting, correcting, recovering, and reducing risk arising from deficient situation awareness (SA) wherein the wireless Smart Phone and wireless Smart Watch is in communication with sport player's coach for reducing and predicting sports player risk arising from deficient situation awareness (SA).

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A computer-based apparatus system for assessing, predicting, correcting, recovering, and reducing risk arising from deficient situation awareness of an individual, said system comprising:
a processor having a memory;
a neurogenic sensor operably disposed adjacent the individual to sense neurogenic and psychophysiological conditions of the individual and provide a neurogenic and psychophysiological data signal;
a data acquisition module in electrical communication with said processor and configured to receive said neurogenic and psychophysiological data signal from said neurogenic sensor;
an individual variable module in electrical communication with said processor, wherein said individual variable module includes at least one of uploaded data corresponding to an individual's baseline situation awareness capacity and artificial intelligence and machine learning neural networks that learn and calibrate to an individual's baseline SA capacity and for determining if one or more predetermined situation awareness deficiency thresholds is exceeded; and
a warning module in electrical communication with said processor configured to activate an alarm in response to said one or more predetermined situation awareness deficiency thresholds being exceeded.

2. A computer-based apparatus system for assessing, predicting, correcting, recovering, and reducing deficient situation awareness according to claim 1, which is further characterized such that said neurogenic sensor is operably disposed in a vehicle and said system is for reducing risk arising from vehicle operator risk.

3. A computer-based apparatus system for assessing, predicting, correcting, recovering, and reducing risk arising from deficient situation awareness according to claim 2, wherein said which includes an autopilot/auto-driver/auto-operator interface in electrical communication with said processor and configured to activate an autopilot/auto-driver/auto-operator/auto-worker/alert/notification command in response to said processor determining that said one or more situation awareness deficiency threshold has been exceeded.

4. A computer-based apparatus system for assessing, predicting, correcting, recovering, and reducing risk arising from deficient situation awareness according to claim 1, wherein said individual variable module employs an AI algorithm Overall Predicted Z_SA=11.646−0.606Z_Vp−0.057Z_Va−0.334Z_Vswm, high task difficulty Predicted Z_SA=11.646−0.277Z_Vp−0.460Z_Va−0.055Z_Vswm, and Low task difficulty Predicted Z_SA=11.646+0.062Z_Vp−0.229Z_Va−0.530Z_Vswm.

6. A computer-based apparatus system for assessing, predicting, correcting, recovering, and reducing risk arising from deficient situation awareness according to claim 1, wherein said neurogenic sensor includes an ear lobe worn sensor.

7. A computer-based apparatus system for assessing, predicting, correcting, recovering, and reducing risk arising from deficient situation awareness according to claim 1, wherein said neurogenic sensor includes a finger worn sensor.

8. A computer-based apparatus system for assessing, predicting, correcting, recovering, and reducing risk arising from deficient situation awareness according to claim 1, wherein said neurogenic sensor includes wrist worn sensor.

9. A computer-based apparatus system for assessing, predicting, correcting, recovering, and reducing risk arising from deficient situation awareness according to claim 1, wherein said neurogenic sensor includes head word sensor.

10. A computer-based apparatus system for assessing, predicting, correcting, recovering, and reducing risk arising from deficient situation awareness according to claim 1, wherein said neurogenic sensor includes a torso worn sensor.

11. A computer-based apparatus system for assessing, predicting, correcting, recovering, and reducing risk arising from deficient situation awareness according to claim 1, wherein said neurogenic sensor includes an individual extremity worn sensor.

12. A computer-based apparatus system for assessing, predicting, correcting, recovering, and reducing risk arising from deficient situation awareness according to claim 2, wherein said neurogenic sensor is operably disposed in a seat of the vehicle.

13. A computer-based apparatus system for assessing, predicting, correcting, recovering, and reducing risk arising from deficient situation awareness according to claim 2, wherein said neurogenic sensor includes is operably disposed in a control stick of said vehicle.

14. A computer-based apparatus system for assessing, predicting, correcting, recovering, and reducing risk arising from deficient situation awareness according to claim 2, wherein said neurogenic sensor is disposed in a in yoke.

15. A computer-based apparatus system for assessing, predicting, correcting, recovering, and reducing risk arising from deficient situation awareness according to claim 2, wherein said neurogenic sensor is operably disposed in a steering wheel.

16. A computer-based apparatus system for assessing, predicting, correcting, recovering, and reducing risk arising from deficient situation awareness according to claim 8, wherein said neurogenic sensor is operably disposed in a smart watch.

17. A computer-based apparatus system for assessing, predicting, correcting, recovering, and reducing risk arising from deficient situation awareness according to claim 1, wherein said neurogenic sensor includes a camera.

18. A computer-based apparatus system for assessing, predicting, correcting, recovering, and reducing risk arising from deficient situation awareness according to claim 1, wherein said neurogenic sensor includes an electroencephalogram apparatus configured to sense predetermined divergence of frequency bands of the individual.

19. A computer-based apparatus system for assessing, predicting, correcting, recovering, and reducing risk arising from deficient situation awareness according to claim 1, wherein said computer-based system includes Smart Phone and Smart Watch.

20. A computer-based apparatus system for assessing, predicting, correcting, recovering, and reducing risk arising from deficient situation awareness according to claim 1 wherein said data acquisition module further includes one of a reflective and a transmissive photoplethysmography apparatus configured to obtain physiological data signal for use in determining said one or more SA level.

21. A computer-based apparatus system for assessing, predicting, correcting, recovering, and reducing risk arising from deficient situation awareness according to claim 1, wherein said the warning module further includes one of a visible indicator, an audible indicator, and a haptic indicator.

22. A computer-based apparatus system for assessing, predicting, correcting, recovering, and reducing risk arising from deficient situation awareness according to claim 2, wherein said warning module is operably connected to one of a smart phone, a satellite and configured to activate an audible indicator, a visual indicator, and a haptic indicator.
